(19) United States
(12) Patent Application Publication
(10) Pub. No.: US 2005/0096369 A1
(43) Pub. Date: May 5, 2005

(54) Compositions and methods for treating cellular proliferation disorders

(76) Inventor: Ba X. Hoang, San Jose, CA (US)

Correspondence Address: PERKINS COIE LLP, P.O. BOX 2168, MENLO PARK, CA, 94026, US

(21) Appl. No.: 10/701,899

(22) Filed: Nov. 4, 2003

(51) Int. Cl. A61K 31/4172; A61K 31/205; A61K 31/155

(52) U.S. Cl. 514/400; 514/554; 514/634; 514/574

(57) ABSTRACT

Compositions and methods for treating patients suffering from a proliferation disorder characterized by an increased voltage gated ion-channel uptake are described. Included are compositions comprised of a compound selected from the group consisting of matrine, oxymatrine, artemisinin, agmatine, and vinpocetine.

### FIELD OF THE INVENTION

The present invention relates to compositions for treatment of a proliferation disorder. The composition comprises matrine, oxymatrine, artemisinin, agmatine, or vinpocetine. The invention also relates to methods of treating a proliferation disorder by modulating the voltage gated ion-channel of the proliferating cells by administering such a composition.

### References

Bond, E. F., AACN Clin Issues, 11(2):261-70 (2000).
Cheema-Dhadli, Eur J Biochem 38:98-102 (1973).
Cho, et al., Planta Medica, 5:343-345, (1986).
Daw, et al., Annu Rev Neurosci., 16:207-22 (1993).
Fairbanks, et al., PNAS, 97(19):10584-10589 (2000).
Fraser, et al., Prostate, 44(1):61-76 (2000).
Derrida, M., TTMA discussion at www.ttma.com/discussion/messages/1592. html.
Guangsheng, Ding, Abstracts of Chinese Medicine, 1(2):287-308 (1987).
Djamgoz home page, www.bio.ic.ac.uk/research/mbad/welcome.htm.
Huang, et al., Sheng Li Xue Bao., 50(2):145-152 (1998).
Ishihara, et al., J Nutr, 130(12):2990-2995 (2000).
Klayman, D L, Science, 228(4703):1049-55 (1985).
Kuhajda, Nutrition, 16(3):202-8, (2000).
Laniado, et al., Prostate, 46(4):262-274 (2001).
Marino, et al., Tumor Biol., 15:82-89 (1994).
Mycielska, et al., J. Cell Physiol., 195(3):461-469 (2003).
Ningxi, Zhu, et al., ACTA Traditional Chinese Medicine and Pharmacology (Shanghai), 15(1):43-44 (2001).
Physician's Desk Reference (PDR) on-line version, www.pdrhealth.com.
Pizer, et al., Cancer Research, (1996) 56(4):745-751.
Ptacek, L. J., Curr Opin Neurol., 11(3):217-26 (1998).
Wang, et al., Zhonghua Zhong Liu Za Zhi., 24(3):230-3 (2002).
Weng, et al., Acta Pharmacol. Sin. (2003) 24(8):746-750.
Wikipedia encyclopedia, on-line version, en.wikipedia.org.
Xiangru, Xu and Jikai, Jiang, Chinese Journal of Integrated Traditional Chinese and Western Medicine, 4(3):235-239 (1998).
Yanxi, Chen, et al., Chinese Journal of Integrated Traditional Chinese and Western Medicine, 22(5):335-336 (2002).
Zhou, et al., JPET, 306:498-504 (2003).

### BACKGROUND OF THE INVENTION

Numerous disease states are characterized by an abnormal ion channel expression. For example, epilepsy, periodic paralyses, and migraine are reported to involve abnormal ion channel expression (Ptacek, 1998 and Bond, 2000). More recently, abnormal voltage gated ion-channels have been associated with metastasis in prostate and lung cancer lines (Mycielska et al., 2003 and Wang et al., 2002 respectively). Metastasis is the process of secondary tumor formation. During metastasis, cancer cells transfer from the primary tumor, enter the circulation (blood or lymph), and migrate around the body to a secondary site. The secondary site may be associated with a specific cancer, e.g. breast cancer tends to spread to lymph nodes and bones. Metastasis can generally be described as a concerted series of basic cellular behaviors, including proliferation, secretion, motility etc.

Ion channel activity is involved in a variety of cellular activities, including cellular proliferation and apoptosis, cell adhesion, cell movement, secretion and even gene expression (Djamgoz home page). Voltage gated ion-channels are a superfamily of proteins present in the membranes that surround all biological cells. These proteins control the flow of ions into and out of cells, and thus help establish the small negative voltage that all cells possess at rest. An ion channel is an integral membrane protein or, more typically, an assembly of several proteins. Such “multi-subunit” assemblies usually involve a circular arrangement of identical or related proteins closely packed around a water-filled pore through the plane of the membrane or lipid bilayer. While large-pore channels permit the passage of ions more or less indiscriminately, the archetypal channel pore is just one or two atoms wide at its narrowest point and conducts a specific species of ion, such as sodium, potassium or calcium. The ions are generally conveyed through the membrane single file, nearly as fast as the ions move through free fluid. Access to the pore is governed by “gates,” which may be opened or closed by chemical or electrical signals, or mechanical force, depending on the variety of channel.

Activation of the ion channels to open or close occurs by various methods. Ion channels can be categorized by their mode of activation into voltage-gated channel, ligand-gated channels, and stretch-activated channels, etc.

Voltage-gated channels such as the voltage gated sodium-channel (VGSC) and the voltage gated potassium channel (VGPC) open or close in response to depolarization or hyperpolarization, respectively. Voltage-gated channels underlie the nerve impulse and because “transmitter-gated” channels mediate conduction across the synapses, these channels are especially prominent in components of the nervous system.

Ligand-gated channels open in response to a specific ligand molecule on the external surface of the cellular membrane in which the channel resides. Examples include the “nicotinic” acetylcholine receptor, AMPA receptor, and other neurotransmitter-gated channels. Cyclic nucleotide-gated channels, calcium-activated channels and others open in response to internal solutes and mediate cellular responses to second messengers.

Stretch-activated channels open or close in response to mechanical forces that arise from local stretching or compression of the membrane around them; for example when their cells swell or shrink. Such channels are believed to underlie touch sensation and the transduction of acoustic vibrations into the sensation of sound.

Certain channels respond to multiple influences. For instance, the NMDA receptor is partially activated by interaction with its ligand, glutamate, but is also voltage-sensitive and only conducts when the membrane is depolarized (Wikipedia). Some calcium-sensitive potassium channels respond to both calcium and depolarization, with an excess of one apparently being sufficient to overcome an absence of the other (Wikipedia).

In eukaryotes, each voltage-gated ion channel (VIC) family channel type has several subtypes based on pharmacological and electrophysiological data. Thus, there are five types of Ca2+ channels (L, N, P, Q and T), at least ten types of K+ channels, each responding in different ways to different stimuli, and at least six types of Na+ channels.

Pharmaceutical agents that modulate ion channels include a benzomorphan derivative, iminodihydroquinolone, tetrodotoxin, and nifedipine. These agents have found application as antiarrhythmic, neuroprotective, and anticonvulsant agents, as well as for local anesthetic uses.

Cancer cells that have the potential to metastasize have “excitable” membranes (Djamgoz home page), similar to nerve and muscle cells. This excitability makes the metastatic cancer cells hyperactive beyond the body's normal control mechanisms. It has been suggested that the “excitable” membranes are due to increased expression of the voltage gated ion-channels in the membranes (Djamgoz home page). A comparison of voltage-gated sodium channel (VGSC) expression in strongly and weakly metastatic prostate lines showed that the VGSC were expressed in the strongly metastatic line, but not the weakly metastatic line (Mycielska et al.). Further, human prostate cancer cell lines showed differential expression of the voltage gated potassium channel (VGPC) in strongly metastatic and weakly metastatic cell lines (Laniado et al. and Fraser et al.). VGSC activity may enhance a variety of cellular behaviors so as to potentiate metastasis. However, VGSC activity had no effect on the cells' proliferation, which, instead, was controlled by VGPC (Fraser et al.).

In addition, many cancers are characterized by expression of elevated fatty acid synthase (FAS) and correspondingly increased fatty acid synthesis and abnormal fatty acid utilization (Pizer et al., 1996). It is proposed that by down-regulating glycolysis, energy available for cellular proliferation may be decreased. Both inhibiting fatty acid synthesis and stimulating oxidative metabolism of fats may further inhibit tumor energy metabolism.

Glycolysis is the sequence of reactions that converts glucose into pyruvate with the concomitant production of a relatively small amount of ATP. Glycolysis can be carried out anerobically (in the absence of oxygen) and is thus an especially important pathway for organisms that can ferment sugars. Glycolysis is the pathway utilized by yeast to produce the alcohol found in beer. Glycolysis also serves as a source of raw materials for the synthesis of other compounds. For example, 3-phosphoglycerate can be converted into serine, while pyruvate can be aerobically degraded by the Krebs or TCA cycle to produce much larger amounts of ATP.

The complete catabolism of glucose includes five stages, divided between anaerobic and aerobic series of reactions. The anaerobic portion of the process, which is the breakdown of glucose to pyruvic acid, is known as glycolysis. Glycolysis is the most ancient series of reactions in the series. Glucose is a stable compound with little tendency to break down spontaneously in to simpler products. If its energy is to be harvested, the glucose must first be made more reactive by the investment of a small amount of energy to “activate” the molecule. The first steps of glycolysis, therefore, are preparatory, enabling the later steps to extract the stored energy.

Many cancers express elevated fatty acid synthase (FAS) and a correspondingly increased fatty acid synthesis and abnormal fatty acid utilization. Recent studies have shown that the FAS inhibitor, cerulenin is selectively cytotoxic to cell lines derived from human malignancies, which suggests that the fatty acid synthesis pathway is a potential target for chemotherapy development (Pizer, etal.).

### SUMMARY OF THE INVENTION

In one aspect, the invention includes a method for treating a person suffering from a cellular proliferation disorder characterized by an increased voltage-gated ion-channel uptake. The method includes administering to the person, a composition containing one or more of matrine, oxymatrine, artemisinin, agmatine, or vinpocetine, or derivatives thereof, in a therapeutic amount. The composition may be administered, for example, on a daily, twice daily or thrice daily schedule, and may be administered on at least a once daily basis for a period of between about 6 months to about 12 months.

The composition may be co-administered with hydroxycitric acid, fumaric acid, or fulvic acid, or salts or derivatives, e.g., esters, thereof. For example, hydroxycitric acid or a derivative thereof may be co-administered at a daily dose of between about 400 mg to about 4000 g, or fumaric acid or a derivative thereof may be co-administered at a daily dose of between about 50 mg to about 2000 mg.

The composition may be given by oral administration and parenteral administration, e.g., intravenous, subcutaneous, intraperitoneal, and intramuscular.

In one exemplary embodiment, the composition administered includes matrine, oxymatrine, or derivatives thereof at a daily dose of between about 5 mg/kg to about 100 mg/kg. In another exemplary embodiment, the composition administered is artemisinin, agmatine, or a derivative thereof, at a daily dose of between about 1 mg/kg to about 20 mg/kg, e.g., at a dose of between about 10 mg/kg to about 20 mg/kg three times daily.

In another aspect, the invention includes composition for treating a cellular proliferation disorder. The composition contains one or more of matrine, oxymatrine, artemisinin, agmatine, vinpocetine, or derivatives thereof; and a physiologically acceptable carrier.

These and other objects and features of the invention will be more fully appreciated when the following detailed description of the invention is read in conjunction with the accompanying drawings.

### BRIEF DESCRIPTION OF THE FIGURES

FIGS. 1A-1E show the structures of the treatment compounds: matrine (FIG. 1A), oxymatrine (FIG. 1B) artemisinin (FIG. 1C), agmatine (FIG. 1D), and vinpocetine (FIG. 1E).

### DETAILED DESCRIPTION OF THE INVENTION

I. Definitions

A proliferation disorder intends a disorder or disease characterized by rapid production of new cells, including but not limited to tumors and metastatic cancer.

The terms “voltage gated ion-channel” and “ion-channel” are used interchangeably and refer to membrane proteins that open and close in response to changes in the electrostatic gradient across the membrane to allow ions into the cell. The ion-channels are ion specific and may include the voltage gated sodium channel (VGSC), voltage gated potassium channel (VGPC) and voltage gated calcium channel (VGCC).

An “enhancing compound” as used herein refers to a compound that down-regulates glycolysis.

The term “treatment” refers to inhibiting or arresting the development of a disease or condition in a patient, particularly a human, causing regression of the disease or condition, or relieving the symptoms associated with the disease or condition.

II. Compositions and Methods for Treatment of Cellular Proliferation Disorders

In one aspect, the invention provides simple, inexpensive, and safe methods and compositions for treating patients suffering from a proliferation disorder, e.g., a condition characterized by uncontrolled proliferation of cells, as occurs in cancer, some autoimmune disorders, and other diseases. Uncontrolled cellular proliferative conditions are often characterized by an abnormal voltage gated ion-channel uptake; thus, the invention provides compositions and methods for treating conditions associated with abnormal voltage-gated ion channel behavior.

Compositions

The composition is one that includes a compound selected from the group consisting of matrine, oxymatrine, artemisinin, agmatine, and vinpocetine. The compound may further be any biologically active form of matrine, oxymatrine, artemisinin, agmatine, and vinpocetine. As will be illustrated below, these compounds have been found to effectively reverse or reduce abnormal cellular proliferation.

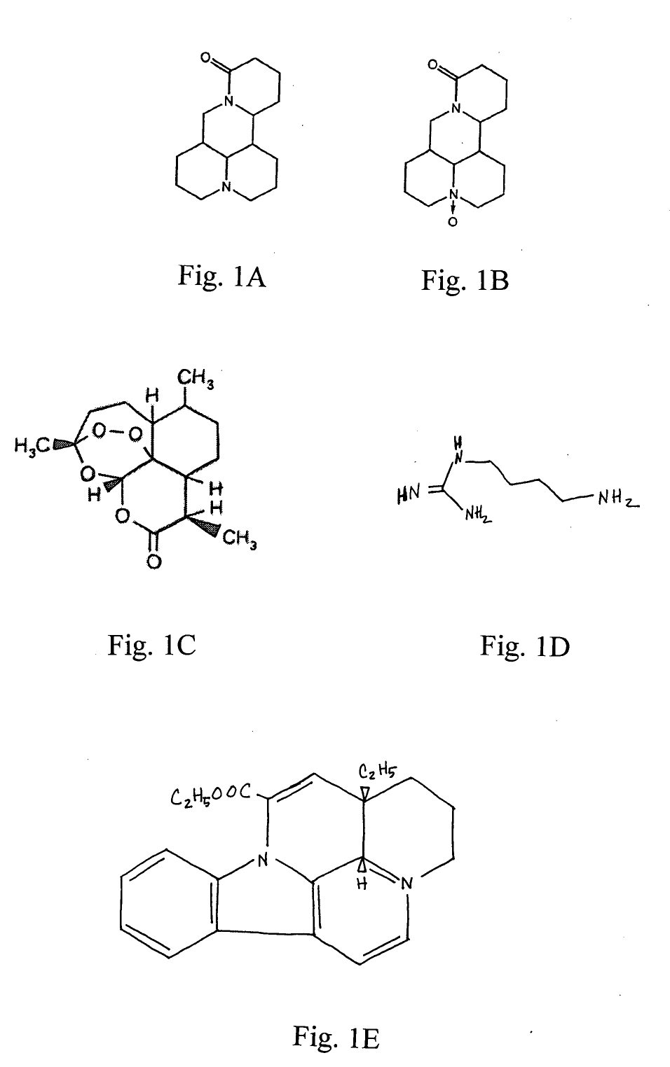

FIGS. 1A-1E show the chemical structures of matrine (FIG. 1A), oxymatrine (FIG. 1B) artemisinin (FIG. 1C), agmatine (FIG. 1D), and vinpocetine (FIG. 1E). Matrine and oxymatrine are the two major alkaloid components found in the roots of species of Sophora, a member of the bean and pea family, Leguminosae. Sophora flavescens naturally contains about 2% of matrine and oxymatrine in the dried root stock and Sophora subprostrata, also called Sophora tonkinensis, contains about 1% of matrine and oxymatrine. Herbs containing some species of Sophora have been used in Chinese folk medicine for a variety of conditions including adjunct cancer treatment, cardiac diseases and arrhythmia, and skin diseases such as psoriasis and eczema. In recent years, oxymatrine has been recommended for treating chronic hepatitis B and hepatitis C (Yanxi, et al.). In a recent pharmacology study, it was reported that matrine could help leukemia cells differentiate into mature and normal white blood cells (Zhu Ningxi, et al.).

As seen from the structures in FIGS. 1A and 1B, matrine and oxymatrine are tetracyclo-quinolizindine alkaloids. Matrine has been obtained in four forms and is water soluble (Merck Index). Matrine is stable in the blood (not converted by the liver) and is excreted by the kidneys within about 24 hours. When taken orally, oxymatrine is converted to matrine, which is considered the more absorbable form. When oxymatrine is administered by injection, it is stable and is excreted as oxymatrine. The toxicity of both matrine and oxymatrine is very low and the central nervous system effects are mild. The LD50 (dose that is lethal to 50% of animals) for injection of a herbal extract (from S. subprostrata) in mice corresponds to a dose of 15 grams of herb per kg (Derrida, 2003), in human terms, this corresponds, roughly, to a single dose of 1 kg of the herb). The LD50 of injected total alkaloids of Sophora subprostrata in dogs is 650 mg/kg (the human equivalent of over 40 grams of alkaloids per day) (Derrida, 2003).

Artemisinin, shown in FIG. 1C, is also known as qinghaosu in China and is extracted from Artemisia annua (sweet woodworm). Artemisia has been used for thousands of years in China to treat malaria (Klayman, 1985). Artemisinin is a sesquiterpene lactone (C₁₅H₂₂O₅, mw 282.3).

Derivatives of artemisinin include artemether, artesunate, arteether, and artelinate. Artemisinin and all the derivatives are quickly converted to the active plasma metabolite dihydroartemisinin. All five artemisinin derivatives inhibit the voltage gated sodium current and intracellular sodium of cells in a dose-dependent manner with the effect being partially reversible (Huang, et al., 1998).

The chemical structure of agmatine is shown in FIG. 1D. This compound is an endogenous neuromodulator present in the brain and spinal cord, and is both a NMDAR antagonist and NOS (nitric oxide synthase) inhibitor. When administered to rodents, it decreases hyperalgesia and inflammation, and normalizes hypersensitivity resulting from nerve injury (Fairbanks, 2000). It is believed to have anti-plasticity and neuroprotective in persistent pain and neuronal injury (Fairbanks et al., 2000). Agmatine is formed by decarboxylation of arginine. Agmatine is an amine and organic cation that is an endogenous ligand at alpha 2-adrenergic and imidazoline (I-) receptors, to which it binds with high affinity.

Agmatine functions as a neurotransmitter and a neuromodulator. Additionally, agmatine inhibits all isoforms of NOS. Agmatine has been shown to block voltage gated calcium channels in cultured rat hippocampal neurons in a concentration and voltage dependant manner (Weng et al., 2003).

Vinpocetine is a semi-synthetic derivative of vincamine, which is an alkaloid derived from the plant Vinca minor L., a member of the periwinkle family. Its chemical structure is shown in FIG. 1D. Vinpocetine, as well as vincamine, are used in Europe, Japan, and Mexico as pharmaceutical agents for the treatment of cerebrovascular and cognitive disorders (PDR). In the United States, vinpocetine is marketed as a dietary supplement called Intelectol® (Covex, Spain). It is sometimes called a nootropic, meaning cognition enhancer, from the Greek noos for mind (PDR).

Vinpocetine is also known as ethyl apovincaminate; ethyl apovincaminoate; eburnamenine-14-carboxylic acid ethyl ester; 3 alpha, 16 alpha-apovincaminic acid ethyl ester; ethyl apovincamin-22-oate; and cavinton.

Vinpocetine has been reported to have calcium-channel blocking activity, as well as voltage-gated sodium channel blocking activity (PDR and Zhou, 2003). It has also been reported to inhibit the acetylcholine release evoked by excitatory amino acids and to protect neurons against excitotoxicity.

Vinpocetine is absorbed from the small intestine, from whence it is transported to the liver via the portal circulation. From the liver via the systemic circulation, it is distributed to various tissues in the body, including the brain. Absorption of vinpocetine is significantly higher when given with food and can be up to about 60% of an ingested dose. On an empty stomach, absorption of an ingested dose can be as low as 7%. Peak plasma levels are obtained one to one and a half hours after ingestion. Extensive metabolism to the inactive apovincaminic acid occurs in the liver. Only small amounts of unmetabolized vinpocetine are excreted in the urine, the major route of excretion of apovincaminic acid. Most of a dose is excreted within 24 hours as this metabolite. The elimination half-life of vinpocetine following ingestion is one to two hours.

In accord with the invention, a composition containing one of the compounds described above is formulated into a preparation suitable for administration to a patient suffering from a cellular proliferation disorder. The composition can be formulated for any desired mode of administration. Solid, liquid, and semi-solid preparations are contemplated and readily prepared by those of skill in the art.

A composition containing matrine, oxymatrine, artemisinin, agmatine, or vinpocetine can additionally include other compounds that offer a therapeutic effect. For example, other compounds that can be included are kava kava and its derivatives, cannibus, graviola, pawpaw, Solanum sp., Sacandrae, Celastrus sp. (including Celastrus hindsii), snake slough, cicada slough, scorpion slough, centipede slough, Crinum latifolium, field bindweed (convulvus), and sedatives.

In studies conducted in support of the invention, compositions comprised of matrine were prepared and administered to test subjects, as set forth in Example 1-7. The test composition used in Examples 1-6 was comprised of equal parts of matrine and cicada slough in powdered form. The test composition used in Example 7 additionally comprised artemisinin. In Examples 5-7, the test composition was administered in combination with an enhancing compound, described further below. In Example 1, a patient suffering from hepatocellular carcinoma, as evidenced by a tumor mass in the liver, was treated with the matrine-cicada slough composition three times daily for a period of two months. At the end of the treatment period, the size of the tumor had decreased significantly.

In another study, detailed in Example 2, a patient suffering from stage IV adenocarcinoma of the pancreas was treated with the matrine-cicada slough composition three times daily for a period of six months. The patient further presented liver and lymphatic nodes metastasis. After two months of treatment, an ultrasound showed no signs of the liver metastasis. After six months of treatment, an ultrasound showed no sign of the pancreatic mass.

A patient suffering from Stage IV ovarian cancer with peritoneal metastasis, as detailed in Example 3, was treated with the matrine-cicada slough composition three times daily for five months. After treatment for five months the ovarian cancer mass and serum CA125 levels were significantly reduced. The patient's peritoneal metastasis was further not present as shown by ultrasound and CT-scan.

As described in Example 4, a patient suffering from metastatic prostate cancer, diffuse bone metastasis, was treated with the matrine-cicada slough composition three times daily. After four months of treatment, the patient's PSA levels were significantly reduced.

In Example 5, a patient suffering from Stage IV lung cancer lymphatic spread, atelectasis, and respiratory failure was treated with the matrine-cicada slough composition in combination with the enhancing compound three times daily. After two months of treatment, X-RAY and CT-scan showed that the tumors were significantly decreased.

As detailed in Example 6, a patient suffering from Stage IV colorectal cancer with lymphatic, soft tissue, lung and liver metastasis was treated with the matrine-cicada slough composition in combination with the enhancing compound twice daily. After six months of therapy, the abdominal cancer was significantly reduced. There was no sign of the lung metastasis and no obvious presence of the liver metastasis.

A patient suffering from breast cancer and metastases in the bone, lung and brain, as detailed in Example 7, was treated with the artemisinin compound in combination with the enhancing compound twice daily. After five months of treatment, a PET scan showed no trace of the breast cancer and the bone metastasis. The brain metastasis was present only as scar traces.

As stated above, the composition of the invention can also include a compound referred to herein as an “enhancing compound”. Enhancing compounds are those capable of down-regulating glycolysis to limit the energy available for cellular proliferation. Compounds suitable for achieving down-regulation of glycolysis include hydroxycitric acid, fumaric acid, and fulvic acid. The enhancing compound may include one or more of the compounds described further below. It will be appreciated that the enhancing compound may further include other compounds that offer a therapeutic effect.

Hydroxycitric acid (HCA) (C₅H₁₄N₄, mw 130) is a compound found in Garcinia cambogia, that has a chemical structure similar to citric acid. The herb has been used for centuries in traditional Hindu medicine for the treatment of chronic indigestion, stomach disorders, rheumatism, etc.

The active ingredient, hydroxyl citric acid, is extracted from the rind of the fruit and has been used as a substance to promote weight loss. Studies have shown that HCA decreases the activity of an essential enzyme in metabolism of fat (Cheema-Dhadli, 1973). The conversion of carbohydrates into fat requires an enzyme called citrate lyase and HCA temporarily reduces the action of this enzyme, blocking the production of fatty acids making less fat available for cellular storage. Thus hydroxycitric acid inhibits synthesis of free fatty acids by inhibiting glycolysis and also stimulating utilization and oxidation of lipid during exercise in mice (Ishihara, 2000). By both inhibiting fatty acid synthesis stimulating oxidative metabolism of fats, HCA inhibits tumor energy metabolism. Hydroxycitric acid thereby breaks the connections between the ATP synthetase and fatty acid synthetase. Sodium and potassium salts of HCA are also useful in embodiments of the invention.

Fumaric acid (C₄H₄O₄, mp 287° C.) is a compound found in small amounts in plants and can be produced synthetically by the distillation of malic acid. Fumaric acid is also formed in the skin during exposure to sunlight. Specifically, fumaric acid is found in Sarcandra Glabra (Thunb) Nakai and Capsella Bursa-pastoris (L). Fumaric acid is a key intermediate in the tricarboxylic acid cycle for organic acid biosynthesis (the KREBS cycle). Chemically, it is an unsaturated dicarbonic acid.

Fumaric acid has been used in food and beverage products since 1946. Fumaric acid is also used in dyes and resins. Fumaric acid has been used with some success to alleviate psoriasis symptoms

Fulvic acid is one of the humic substances, which are ubiquitous in nature and arise from the decay of plant and animal residues in the environment. Humic substances are divided into humic acid and fulvic acid on the basis of water solubility as a function of pH. Fulvic acid is the fraction that is soluble in water under all pH conditions and is in general lower in molecular size and weight and lower in color intensity than humic acids.

Humic substances commonly account for 50% of the dissolved organic carbon concentrations in stream water, of which 90 to 95% are fulvic acids. Humic acids are 3 to 5 times more abundant in soils than fulvic acids (Stevenson, 1982), whereas fulvic acids are 9 to 10 times more abundant in water than humic acids (Malcolm, 1985).

Fulvic acids from peat have been found to scavenge both superoxide and hydroxyl radical (Wang et al, 1966). It has also been shown that fulvic acids prevent the absorption of mutagens through the rat small intestine using a highly mutagenic furanone found in chlorinated water and an in vitro everted rat gut sac system (Clark and Chipman, 1995). Although the presence of fulvic acids in the drinking water of certain parts of China has been coupled to the incidence of Kashin-Beck disease, this only occurred in conjunction with a selenium deficient diet (Peng and Xu, 1987).

Compositions comprising fulvic acid in the form of a salt, an ester, or a derivative that retains activity are contemplated. Fulvic acid may be derived from a wet coal oxidation process of the type described in U.S. Pat. No. 4,912,256.

Methods of Treatment

A. Treatment of Cellular Proliferation and Decreasing Voltage Gated Ion-channel Activity

The invention also provides a method for treating a patient suffering from a cellular proliferation disorder, particularly from cellular proliferation disorders characterized by an increased voltage-gated ion-channel uptake. Symptoms associated with such conditions are treated or alleviated by the method described herein.

Compositions are prepared to contain one of matrine, oxymatrine, artemisinin, agmatine, or vinpocetine, and, optionally, an enhancing compound, such as hydroxycitric acid, fumaric acid, and fulvic acid, and/or other compounds with therapeutic benefit are administered to the patient by any desired route, discussed in more detail below. As illustrated by the studies set forth in Examples 1-7, discussed above, the compositions effectively arrest uncontrolled cellular proliferation and cause a reduction in cellular masses, such as tumors. Accordingly, the invention contemplates a method of treating conditions of cellular proliferation by administering a composition comprising one of the recited compounds in an amount effective to arrest uncontrolled cellular proliferation.

The invention also contemplates a method of treating cellular proliferation disorders characterized by an abnormal ion-channel uptake. The role of ion channels in some forms of cancer, such as breast cancer (Marino et al., 1994) and prostate cancer (Mycielska et al., 2003) has been shown. Stabilizing and/or normalizing the action and/or hyperactivity of sodium and potassium, calcium, or other mineral ion pumps with ion channel modulators or blockers may cause reversion to a non-cancerous state. Additionally, the hyperactivity and increased number of voltage gated ion-channels in proliferating cells is likely a compensatory survival mechanism of the cells that may cause inflammation, pain, and uncontrolled proliferation. The localized and whole-body pain associated with cancer may also be associated with an increase in voltage gated ion-channel activity.

Thus, a therapeutic method for altering or controlling ion-channel activity directly impacts the cellular proliferation and its symptoms. The effect of a composition prepared according to the teachings provided herein on the ion-channel activity can be determined using in vitro and in vivo techniques. For example, voltage gated ion-channel activity can be measured using the voltage-clamp method. This method relies on the fact that the ions flow from one side of the cell membrane to the other via the voltage gated ion channels. Undesired capacitative currents flow when the membrane potential changes. A device called a voltage-clamp amplifier (e.g. an Axopatch 200 Amplifier available from Axon Instruments, Foster City, Calif.) can depolarize a cell to some specified potential, and then via a feedback amplifier prevent the cell's membrane potential from deviating from that specified potential. In response to a depolarizing command under voltage-clamp a rapid inward current is followed with short latency by a sustained outward current. The amount of current it takes to hold the membrane at this new voltage (against the flow of these ions) is directly related to the number of channels opened. This technique allows the activity of ion channels to be studied at a constant membrane potential, enabling their properties to be more easily quantified. The effect of a compound as a potential modulator of channel activity is readily determined by examining the cellular behavior in the presence and absence of the compound or composition. Exemplary voltage clamp protocols are presented in Laniado, et aL (Prostate, 46(4):262-272, 2001), which is incorporated herein by reference.

The method of the invention is further useful for treating or reducing the symptoms associated with asthma, inflammatory bowel diseases, insomnia, seizures, hyperactivity, and chronic pain. The method of the invention is further useful for treating conditions associated with increased activity of the voltage gated ion-channel associated or caused by stress, toxic chemicals, and hypoxia-ischemia.

B. Down-regulation of Glycolysis

As indicated above, many cancers express elevated fatty acid synthase (FAS) and correspondingly increased fatty acid synthesis and abnormal fatty acid utilization (Kuhajda, 2000). It is an embodiment of this invention to co-administer compounds to inhibit glycolysis and ATP production. It is theorized that cellular proliferation disorders characterized by an increased voltage-gated ion-channel uptake may additionally be characterized by an energy deficient state. These disorders may exhibit elevated FAS and abnormal fatty acid utilization as indicated above. It is proposed that by down-regulating glycolysis, energy available for cellular proliferation may be decreased. Both inhibiting fatty acid synthesis and stimulating oxidative metabolism of fats may further inhibit tumor energy metabolism.

Recent studies have shown that cerulenin, a FAS inhibitor, is selectively cytotoxic to cell lines derived from human malignancies. This data suggests that the fatty acid synthesis pathway is a potential target for chemotherapy development (Pizer et al. 1996). Malignant cells can retain dependence on endogenous fatty acid levels supports the notion that FAS inhibitors may be useful in treating cancer in vivo.

As described in Examples 5-7, one or more enhancing compounds may optionally be co-administered to down-regulate over stimulated glycolysis. As described above several compounds have been identified that mediate glycolysis and/or fatty acid production. The enhancing compound may be administered before, after or concurrently with the therapeutic composition. The therapeutic compound may further be formulated to include the enhancing compound.

C. Routes of Administration and Dosages

Matrine and oxymatrine may be isolated from the herbs Sophora tonkinensis (Sophora subprostrata) or Sophora flavensis. Matrine and oxymatrine may be extracted from the herb by any known methods. Preferably, 5 to 100 mg/kg of matrine and/or oxymatrine are administered one, two or three times daily. In another embodiment, 10 to 100 mg/kg of matrine and/or oxymatrine are administered one two, or three times daily. In yet another embodiment, the crude, dried herb may be administered directly. In this embodiment, 2 to 30 grams of the crude, dried herb is administered daily. In a preferred embodiment, 15 grams of crude, dried Sophora tonkinensis or Sophora flavensis is administered. It will be appreciated that derivatives of matrine and oxymatrine that retain the desired therapeutic activity are suitable for administration in this method.

Artemisinin may be extracted from Artemisia annua (also known as Qinghao or sweet annie). Agmatine may be formed by decarboxylating arginine, or may be isolated from several plant and animal sources, e.g., pollen, ergot, herring sperm, octopus muscle. Artemisinin, agmatine and/or suitable derivatives are preferably administered at about 1 mg/kg to 20 mg/kg one to three times daily. It will be appreciated that derivatives of artemisinin and agmatine that retain the desired therapeutic activity are suitable for administration in this method.

Vinpocetine may be extracted from Vinca minor (periwinkle plant). Vinpocetine and/or suitable derivatives are preferably administered at about 10 to about 100 mg daily, more preferably about 15 mg to about 100 mg daily. In one embodiment, about 10 mg to about 20 mg of vinpocetine is administered three times daily. It will be appreciated that derivatives of vinpocetine that retain the desired therapeutic activity are suitable for administration in this method.

Hydroxycitric acid may be isolated from the fruit or rind of Garcinia cambogia. Hydroxycitric acid may be isolated from the herb by any known methods. In one embodiment, a sodium or potassium salt of hydroxycitric acid is administered. In a preferred embodiment, about 400 mg to about 4000 g of hydroxycitric acid is administered daily. In another embodiment, about 400 mg to about 4000 mg of hydroxycitric acid is administered daily. In another embodiment, the fruit or rind of the herb is administered directly. In this embodiment, 2 to 4 grams of the herb is administered 2-3 times daily. It will be appreciated that derivatives of hydroxycitric acid, including, but not limited to sodium or potassium salts, that retain the desired therapeutic activity are suitable for administration in this method.

Fumaric acid may be isolated from Sarcandra glabra or Capsella bursa pastoris. Fumaric acid may also be produced synthetically. It will be appreciated that derivatives of fumaric acid, including fumaric acid in ester form, that retain the desired therapeutic activity are suitable for administration in this method. In one embodiment, dimethylfumarate is administered. In a preferred embodiment, 50 mg to 2000 mg of an ester form is administered daily. In another embodiment, the crude, dried herb may be administered directly. In this embodiment, 15-30 grams of the crude, dried herb is administered daily.

It will be appreciated that any of the above compounds described may be administered in isolated/extracted form or as the crude herb. It will further be appreciated, as detailed in Example 7, the treatment composition may include one or more of matrine, oxymatrine, artemisinin, agmatine, vinpocetine, and derivatives thereof. One of skill in the art will further appreciate that the enhancing compound may comprise one or more of hydroxycitric acid, a sodium or potassium salt of hydroxycitric acid, fumaric acid, fulvic acid, or derivatives thereof, as detailed in Examples 5-7.

Routes of delivery include, but are not limited to, various systemic routes, including oral and parenteral routes, e.g., intravenous, subcutaneous, intraperitoneal, and intramuscular, as well as inhalation and transdermal delivery. Administration via these routes is achieved by formulating the compositions into a suitable dosage form. Non-limiting examples include pills, tablets, capsules, suspensions, syrups, buccal/mucosal patches, gels, ointments, suppositories, and the like. Preparation of such dosage forms is routine to those of skill in the art and exemplary references describing preparation of extracts, decoctions, pills, and suspensions are known, such as Chinese Herbal Medicine: Materia Medica; Dan Bensky and Andrew Gamble, ed.; Eastland Press, Seattle, c1986, which is incorporated herein in its entirety. In a preferred embodiment, the composition is administered orally.

Parenteral administration includes injection or gradual infusion over time. The compounds of the invention can be injected intravenously, intraperitoneally, intramuscularly, intratumorally, intranasal or administered transdermally.

The composition may be administered directly to a subject or in a suitable pharmaceutical carrier. In one embodiment, the composition is administered with a physiologically acceptable carrier, excipient, or diluent, where the composition is dissolved or dispersed therein as an active ingredient and formulated according to conventional practice. The carrier may be any of a variety of standard physiologically acceptable carrier employed by those of ordinary skill in the art. It will be understood that the choice of suitable physiologically acceptable carrier will vary dependent upon the chosen mode of administration.

Sustained release compositions are also contemplated within the scope of this application. These may include semipermeable polymeric matrices in the form of shaped articles such as films or microcapsules.

In one embodiment, the composition may be administered at regular intervals, e.g., daily, two times daily or three times daily. In another embodiment, the composition is administered over a period of time, e.g. 6 to 12 months or more. It will be appreciated that administration of the composition may be continued for an indefinite time period. It will further be appreciated that the schedule and time period for administration of the therapeutic composition and the enhancing compound may differ e.g. the therapeutic composition is administered thrice daily and the enhancing compound is administered twice daily. Additionally, the therapeutic composition may be administered for a longer or shorter time period as the enhancing compound. The enhancing compound may further be administered intermittently throughout the administration of the therapeutic composition.

It will be appreciated that dosages of the composition will vary dependent upon the compound used in the composition. Preferred doses for oral administration of matrine and oxymatrine are from about 10 mg/kg to 100 mg/kg on a daily basis. Preferred doses for oral administration of artemisinin and agmatine are from about 1 mg/kg to about 20 mg/kg on a daily basis. Preferred doses for oral administration of vinpocetine are from about 15 to about 100 mg daily, more preferably about 10 mg to about 20 mg, three times daily.

Dosages will vary in accordance with such factors as the age, health, sex, size and weight of the patient, the route of administration, and the efficacy of the compound with respect to the voltage gated ion-channel. Greater or lesser amounts of the compound may be administered as required.

It will be appreciated that the treatment composition and/or the enhancing composition may contain one or more of the described compounds. It will further be appreciated that other herbs or ingredients may be administered with the treatment compound or the enhancing compound. For example, cicada slough, scorpion slough, snake slough may be mixed with at least one of matrine, oxymatrine, artemisinin, agmatine, and vinpocetine to form the treatment compound. Cicada Slough is the slough of Cryptotympana pustulata Fabricius (Family Cicadidae) dropped off the nymph during emergence.

### III. EXAMPLES

### Materials and Methods

Preparation of Treatment Compositions

The powdered matrine treatment composition in the following examples consisted of an equal mixture of matrine and cicada slough (snake slough, Centipede slough, scorpion slough may be used). In capsule form, the treatment compound consists of 120 mg matrine and 120 mg cicada slough. Matrine may be isolated from the root of Sophora sp. by any known methods in the art. An exemplary method is described in Cho etal. Planta Medica, 5:343-345, (1986).

The enhancing compound in the following examples consisted of 1000 mg of hydroxycitric acid and 100 mg of dimethylfumarate, an ester of fumaric acid. Hydroxycitric acid may be isolated from the fruit or rind of Garcinia cambogia by any known methods in the art. Fumaric acid may be isolated from Sarcandra glabra or Capsella bursa pastoris. Exemplary methods are described in Cho et al. Planta Medica, 5:343-345, (1986).

Alkaline Phosphatase (ALP) Assay

Alkaline phosphatase is an enzyme found in all tissues. Tissues with particularly high concentrations include liver, bile ducts, placenta and bone. Since damaged or diseased tissue releases enzymes into the blood, serum ALP measurements can be abnormal in many conditions, including bone disease and liver disease. The normal range is 44 to 147 IU/L (international units per liter).

Prostate Specific Antigen (PSA) Assay

Prostate specific antigen (PSA) is a 34 kDa 240 amino-acid glycoprotein produced exclusively by prostatic epithelial cells. Elevated levels of serum PSA are associated with prostate pathologies including prostate cancer. Normal levels of PSA for an average man ranges from 0 to 4 (ng/ml). A PSA level of 4 to 10 ng/ml is considered slightly elevated; levels between 10 and 20 ng/ml are considered moderately elevated; and levels above 20 ng/ml are considered highly elevated.

Alpha Fetoprotein (AFP) Assay

Alpha fetoprotein (AFP) is a protein normally produced by the liver and yolk sac of a fetus and AFP levels decrease soon after birth. Levels of AFP in serum are measured to diagnose or monitor fetal distress or fetal abnormalities; diagnose some liver disorders; and to screen for and monitor some cancers. Normal serum values of AFP in males or nonpregnant females are less than 10 micrograms/milliliter. Greater-than-normal levels of AFP may indicate cancer.

CA125 Assay

CA125 is a mucin-like molecule that is produced by mesothelial cells of the peritoneum and endometrium. Serum levels of CA125 are elevated in more than 80% of women with epithelial ovarian cancer. The association of elevated serum concentrations of CA125 and endometriosis is more marked in individuals with Stage IlIl and IV compared with Stage I and II endometriosis. One method of detecting CA125 is by the Chiron Diagnostics ACS: 180 OV assay, which is a two-site sandwich immunoassay using direct chemiluminscence technology.

The CA 125 test measures a sugar protein that may be released when cells are inflamed or damaged. CA 125 levels are usually measured by a blood test, but can also be detected in fluid from the abdominal and chest cavities. Levels under 35 kU/ml are considered normal. Ovarian cancer cells may produce an excess of these protein molecules, as may some other cancers, including cancer of the fallopian tube or endometrial cancer (cancer of the lining of the uterus).

### Example 1

### In vivo Administration of Composition for Treatment of Hepatocellular Carcinoma

A 47 year-old female was diagnosed with hepatocellular carcinoma, Hepatitis B and cirrhosis. An ultrasound showed an 8 cm and a 12 cm mass in the right lobe of the liver. The serum alpha fetoprotein (AFP) level was determined to be 96 ng/ml (normal serum levels are up to 10 ng/ml). Greater than normal serum AFP levels may indicate cancer or cirrhosis. A CT-guided biopsy was performed to confirm a diagnosis of hepatocellular carcinoma and cirrhosis.

The patient was treated with two teaspoons of the treatment compound in powdered form (600 mg matrine and 600 mg cicada slough) three times daily. After two months of treatment, a CT-scan showed a decrease in tumor size of greater than 60%. Serum AFP level was reduced to 36 ng/ml.

The treatment was increased to three teaspoons of the treatment compound in powdered form, three times daily. At four months, a CT-scan showed the tumor size decreased 1 cm and 2 cm, respectively. Serum AFP levels dropped to 11 ng/ml.

### Example 2

### In vivo Administration of Composition for Treatment of Adenocarcinoma

A 65 year-old female was diagnosed with Stage IV invasive adenocarcinoma of the pancreas. An ultrasound showed a 4 cm×6 cm pancreatic mass. The patient further presented liver and lymphatic nodes metastasis.

The patient was treated with three teaspoons, three times daily of a mixture containing equal amounts of matrine and cicada slough in powdered form. The patient reported disappearance of leg edema and associated pain within 17 days. After two months of treatment, an ultrasound showed the pancreatic mass to be 2 cm×2 cm with no sign of the liver metastasis. After six months of treatment, an ultrasound showed no sign of the pancreatic mass.

### Example 3

### In vivo Administration of Composition for Treatment of Ovarian Cancer

A 72 year-old patient was diagnosed by CT-scan with Stage IV ovarian cancer diagnosed. Ultrasound and pathologic examination showed numerous 2-3 cm peritoneal metastasis and a 9 cm×8 cm ovarian cancer mass. The serum CA125 (a marker for ovarian cancer) was 2,702 units/mi (levels are considered normal up to 35 units/ml).

The patient was treated with two teaspoons of the treatment compound (600 mg matrine and 600 mg cicada slough) three times daily. After two months of treatment, serum CA125 level was 1038 units/ml. A CT-scan revealed a reduction of tumor size by more than 50%. After five months of treatment, an ultrasound and CT-scan showed no signs of the peritoneal metastasis and a 2 cm×3 cm ovarian cancer mass. Serum CA125 level was 42 units/ml.

### Example 4

### In vivo Administration of Composition for Treatment of Prostate Cancer

A 59 year-old man was diagnosed with metastatic prostate cancer had a first serum PSA level of 196 ng/ml. The bone scan showed diffuse bone metastases. A prostate biopsy showed Gleason 9/10 adenocarcinoma in two out of six cores. The alkaline phosphatase level was 1962 IU/L. The patient additionally experienced significant bone pains.

The patient was treated with two teaspoons of the treatment compound (600 mg matrine and 600 mg cicada slough) three times daily. After ten days of treatment, the patient reported a decrease in bone pain, however serum PSA levels had increased to 278 ng/ml. In two months, the PSA dropped transiently to 19 ng/ml. After four months of therapy, the PSA level was 5.7 ng/ml.

### Example 5

### In vivo Administration of Composition for Treatment of Lung Cancer

A 47 year-old Asian man developed a severe cough and was treated with cough medicine and antibiotics. A X-RAY showed a significant enlargement in the right lung. Two months later, a second X-RAY and a CT-scan showed a tumor (11×7 cm) close to the heart and multiple tumors in both lung and lymphatic nodes. The patient also exhibited physical and clinical symptoms of respiratory failure. The patient was diagnosed with Stage IV lung cancer lymphatic spread, atelectasis, and respiratory failure.

Matrine and cicada slough was administered as six capsules (120 mg matrine and 120 mg cicada slough per capsule), three times daily. Two teaspoons of the enhancing compound (1000 mg of hydroxycitric acid and 100 mg of dimethylfumarate) was co-administered three times daily. After two months, an X-RAY showed minimal scars in the lung, but no masses. A CT-scan confirmed reduction of the tumors.

### Example 6

### In vivo Administration of Composition for Treatment of Rectal Cancer

A 62 year-old female was diagnosed with rectal cancer. Surgery was performed to remove the tumor followed by three months of chemotherapy and radiation therapy.

Eight months later, the patient had a colon obstruction. A CT-scan confirmed multiple abdominal tumors in and outside of the intestines as well as liver and lung metastasis. Symptoms included pain, extreme nausea, ascites and enlargement of abdominal and esophagus veins.

The patient was diagnosed with Stage IV colorectal cancers with lymphatic, soft tissue, lung and liver metastasis.

Matrine and cicada slough was administered as six capsules (120 mg matrine and 120 mg cicada slough per capsule), twice daily. The enhancing compound (1000 mg of hydroxycitric acid and 100 mg of dimethylfumarate) was co-administered twice daily. After six months of therapy, a CT-scan shown an 80% reduction of cancer in the abdomen. There were no signs of lung metastasis and there were three small scar spots in the liver without obvious presence of cancer.

### Example 7

### In vivo Administration of Composition for Treatment of Breast Cancer

A 54 year-old female was diagnosed with breast cancer and metastases in bone, lung and brain.

Matrine and cicada slough was administered as five capsules (120 mg matrine and 120 mg cicada slough per capsule), three times daily. Artemisinin (200 mg) was administered three times daily. Three teaspoons of the enhancing compound (1000 mg of hydroxycitric acid and 100 mg of dimethylfumarate) was co-administered twice daily. After five months of treatment, a PET scan showed no activity anywhere in the breast, chest, bone and only scar traces in the brain. An additional scan after an additional seven months did not confirm cancer activity anywhere in the patient's body.

Although the invention has been described with respect to particular embodiments, it will be apparent to those skilled in the art that various changes and modifications can be made without departing from the invention.

1. A method for treating a person suffering from a cellular proliferation disorder characterized by an increased voltage-gated ion-channel uptake, comprising:
administering to the person a composition comprised of a compound selected from the group consisting of matrine, oxymatrine, artemisinin, agmatine, and vinpocetine.

2. The method of claim 1, wherein said administering is performed on a schedule selected from daily administration, twice daily administration, and thrice daily administration.

3. The method of claim 1, wherein said administering is performed at least once daily for a period of between about 6 months to about 12 months.

4. The method of claim 1, further comprising:
co-administering an enhancing compound selected from the group consisting of hydroxycitric acid, a sodium or potassium salt of hydroxycitric acid, fumaric acid, an ester of fumaric acid, fulvic acid, and a salt of fulvic acid.

5. The method of claim 4, when said co-administering includes co-administering hydroxycitric acid or a derivative thereof at a daily dose of between about 400 mg to about 4000 g.

6. The method of claim 4, when co-administering includes co-administering fumaric acid or a derivative thereof at a daily dose of between about 50 mg to about 2000 mg.

7. The method of claim 1, wherein said administering is selected from the group consisting of oral administration and parenteral administration.

8. The method of claim 1, wherein said administering includes administering parenterally via a parenteral route selected from the group consisting of intravenous, subcutaneous, intraperitoneal, and intramuscular.

9. The method of claim 1, wherein said administering includes administering a compound selected from matrine, oxymatrine, and a derivative thereof, at a daily dose of between about 5 mg/kg to about 100 mg/kg.

10. The method of claim 1, wherein said administering includes administering a compound selected from artemisinin, agmatine, or a derivative thereof at a daily dose of between about 1 mg/kg to about 20 mg/kg.

11. The method of claim 1, wherein said administering includes administering vinpocetine at a dose of between about 10 mg/kg to about 20 mg/kg three times daily.

12. A method of treating a patient suffering from a cellular proliferation disorder, comprising:
administering to the patient a composition comprised of a compound selected from the group consisting of matrine, oxymatrine, artemisinin, agmatine, and vinpocetine.

13. A composition for treating a cellular proliferation disorder, comprising:
a compound selected from the group consisting of matrine, oxymatrine, artemisinin, agmatine, vinpocetine, and derivatives thereof; and
a physiologically acceptable carrier.
